(19) United States
(12) Patent Application Publication
(10) Pub. No.: US 2014/0076332 A1
(43) Pub. Date: Mar. 20, 2014

(54) Dental Appliance for Treatment of Obstructive Sleep Apnea (OSA) and Sleep Bruxism

(71) Applicant: Kenneth Luco, Kingston (CA)

(72) Inventor: Kenneth Luco, Kingston (CA)

(21) Appl. No.: 13/621,187

(22) Filed: Sep. 15, 2012

(51) Int. Cl. A61F 5/56

(52) U.S. Cl. CPC A61F 5/566
USPC 128/848

(57) ABSTRACT

A sleep apnea device having top and bottom trays, as well as a method of use thereof, is disclosed herein. Each tray is adapted to be retained on respective top and bottom teeth, the bottom tray having two flanges extending upwards from a generally planar side of the bottom tray, opposite a side which abuts the teeth. As such, when worn, the flanges extend toward the top tray. The top tray has corresponding flat regions on each side thereof which cut into the outside (relative to the teeth) plane of the tray. The flanges thus abut such flat regions on the outer side of the upper tray, pushing the lower tray and, therefore, lower teeth and tongue forward. A wearer may then sleep wearing the trays, prevent obstruction of the airways by the tongue during sleep.

### FIELD OF THE DISCLOSED TECHNOLOGY

The disclosed technology relates generally to an oral appliance worn at night for obstructive sleep apnea treatment. More specifically, the disclosed technology relates to an oral appliance which repositions the lower jaw forward, to push the tongue forward (mandibular advancement).

### BACKGROUND OF THE DISCLOSED TECHNOLOGY

There are three forms of sleep apnea: central (CSA), obstructive (OSA), and complex or mixed sleep apnea (i.e., a combination of central and obstructive) constituting 0.4%, 84% and 15% of cases, respectively. In CSA, breathing is interrupted by a lack of respiratory effort; in OSA, breathing is interrupted by a physical block to airflow despite respiratory effort, and snoring is common. Referring now to OSA, more than 12 million American adults have obstructive sleep apnea. It is a disorder in which breathing is repeatedly, but briefly, interrupted during sleep. Obstructive sleep apnea occurs when the muscles in the back of the throat fail to keep the airway open, making it difficult to breathe. It is more common in people who are overweight, but it can affect anyone. For example, some small children may have obstructive sleep apnea due to enlarged tonsil tissue in their throats. For those with OSA, the airway collapses or becomes blocked during sleep. This causes shallow breathing, or pauses in breathing, which leads to fragmented sleep and low blood oxygen levels. OSA is commonly accompanied by snoring, as well. This is referred to as Upper Airway Restrictive Syndrome (UARS) and may also occur from medical conditions such as deviated septum, large turbinates, narrow palate, long soft palate etc. This condition is considered to be the same as OSA and is diagnosed and treated similarly. sleep bruxism is another sleep disorder that occurs concurrently with OSA in a majority of patients. This is forceful side to side, front to back or vertical clenching and unclenching of the mandible. This is seen in all age groups.

Some patients with obstructive or other types of sleep apnea may benefit from surgery. The type of surgery depends on the cause of the sleep apnea. Examples for common types of surgical procedures for sleep apnea include removing the tonsils and adenoids, if these organs are blocking the airway. This can be especially helpful in the case of children. In advanced, non-responsive cases, the mandible may be broken and lengthened, to move the tongue out of the throat region. Another surgery involves placing a small hole and tube in the windpipe, causing airflow through the tube into the lungs. This is carried out in severe cases of sleep apnea. In still other cases, the uvula (the tissue that hangs down in the middle of the back of the throat), and part of the roof of the mouth at the back of the throat may be removed.

Another common treatment for sleep apnea, which does not require invasive surgery, is called continuous positive airway pressure (CPAP). In this treatment, the patient wears a special mask over nose and mouth (or inserts into the nose) while sleeping. The mask keeps the wearer's airway open by increasing the air pressure breathed in by the wearer. However, such masks cover a large portion of the face and head, are generally uncomfortable, and may leak, can fall off, thus reducing effectiveness. They do not treat Sleep Bruxism.

Still further, devices of the prior art have been designed to aid in obstructive sleep apnea problems. U.S. Pat. No. 5,427,117, issued to Thornton et al., discloses a dental device including adjustable upper and lower arch trays. The device has an upper arch and lower arch with an adjustable post that extends from the upper arch and contacts the lower arch, so that the user's lower jaw is extended forward with respect to the upper. The post shown in these references is shaped to engage with the lower arch, so that the user's mouth cannot be opened more than a predetermined amount while the post is engaged.

Still another device has been described in U.S. Pat. No. 5,829,441 issued to Kidd et al., which discloses a mandible extension dental device including adjustable upper and lower arch trays. Each upper and lower arch tray includes a U-shaped tray body attached to the upper and lower teeth. The device is adjusted to selectively cause extension of the user's lower jaw when the mouth is closed. The upper and lower trays dovetail with a depending hook located in the front of the device.

U.S. Pat. No. 6,766,802, issued to Keropian et al., discloses an appliance that covers the inside of the upper teeth only with an open palate similar to an upper orthodontic retainer. This device further has a retainer wire to hold the anterior teeth in place, or to move such teeth backwards, and a raised strip that extends from an incisor tip (biting edge) of central incisors to separate the posterior teeth and hold the tongue down.

Thus, while there are many different ways of attempting to alleviate problems associated with OSA, there is still room for improvement. There is a need in the field to find devices which are least obtrusive, most comfortable, easy to use, durable, and most effective.

### SUMMARY OF THE DISCLOSED TECHNOLOGY

Therefore, an object of the disclosed technology is to provide a device and method of use of the device to prevent obstructive sleep apnea and sleep bruxism.

A further object is to provide such a device which keeps the lower jaw forward during sleep, to prevent the tongue from falling backward.

In an embodiment of the disclosed technology, separate upper and lower trays of a retainer device adapted to abut or attach around respective top and bottom teeth are used. The upper tray has at least two mounting regions with adjoined outer, inner, and lower portions adapted to abut corresponding portions of the upper teeth. Each such outer portion of each mounting region of the upper tray further has a generally flat front portion defining a front area of the mounting region. This generally flat front portion is usually perpendicular to the plane of the outer side of the mounting region and forms a corner there-on. The lower tray has at least two mounting regions with adjoined outer, inner, and upper portions adapted to abut corresponding portions of lower teeth. A flange extending from each of two such mounting regions of the lower tray, extends generally away from the lower teeth, and the front of each flange defines the front-most extremity of each mounting region. In embodiments of the disclosed technology, the flange(s) is/are adapted to abut the generally perpendicular portion of the mounting region(s) of the upper tray, and when the flange abuts in such a manner and the trays are worn/abutted against the bottom teeth, these teeth are forced forward relative to a resting portion of the teeth. As such, it follows that the tongue is held forward compared to its resting position.

In embodiments of the disclosed technology, the lower side of the mounting region of the upper tray, and the upper side of the mounting region of the lower tray are adapted to abut one another along a length thereof, extending over respective adjacent sides of a plurality of teeth. The flange extends diagonally upwards from the lower tray, to the outside of the upper tray, in embodiments of the disclosed technology. Such a diagonal or angle of extension of the flange may be between 60 and 80 degrees, relative to the length of the mounting region which extends over the adjacent sides of the plurality of teeth.

The side perpendicular to the flat portion of the upper tray further forms a side of an adjustable block. This adjustable block (e.g., block 132/142) further has an adjustable length attachment, adjustably attaching the adjustable block to a rear portion (defined as the portion behind the adjustable block) of the mounting portion of the upper tray, such that the adjustable block is movable closer to the rear portion by way of the adjustable length attachment. The adjustable length attachment may be a screw turnable by inserting a wire key.

The flange may abut both the adjustable block and the generally flat front portion of the upper tray when worn by a user. Further, on each tray, the mounting regions may be connected to each other by a wire adapted to abut the front sides of teeth situated between the mounting regions, and the mounting regions may be defined, when using such a wire, as the portions which are adapted to fit over the back teeth and connect to the wire. These wires act to minimized shifting of the front teeth when worn.

In a method of treating obstructive sleep apnea with a two piece oral appliance, one dons a lower tray of the oral appliance with an inner surface abutting lower teeth, and an outer surface opposite the inner surface, the outer surface further having two flanges extending upwards from the outer surface. One also dons an upper tray of the oral appliance with an inner surface abutting upper teeth, and an outer surface opposite the inner surface, the outer surface being generally planar and interrupted by a surface which is generally perpendicular to the generally planar surface. Then, one abuts the two flanges of the lower tray against the generally perpendicular surface the outer surface of the upper tray. This causes, in an embodiment of the disclosed technology, the lower teeth to be held in front of said upper teeth, relative to a resting position of said teeth. The wearer of the two piece oral appliance sleeps with said oral appliance donned on his or her teeth, in an embodiment of the disclosed technology.

In another embodiment of the disclosed technology, a two piece oral appliance device for treating obstructive sleep apnea has a lower tray with an inner surface abutting lower teeth, and an outer surface opposite the inner surface, the outer surface further having two flanges extending upwards from the outer surface. An upper tray of the oral appliance has an inner surface abutting upper teeth, and an outer surface opposite the inner surface, the outer surface being generally planar and interrupted by a surface which is generally perpendicular to the generally planar surface. The two flanges are abutted, each to one of the generally perpendicular surfaces of the outer surface of the upper tray. Such abutment, in embodiments of the disclosed technology, causes the lower teeth to be held in front of the upper teeth, relative to a resting position of the teeth.

### BRIEF DESCRIPTION OF THE DRAWINGS

FIG. 1 shows a lower perspective view of an upper tray and upper perspective view of a lower tray to show how the two trays fit together in an embodiment of the disclosed technology.

FIG. 2 shows a top perspective view of the bottom tray of FIG. 1.

FIG. 3 shows a bottom perspective view of the top tray of FIG. 1.

FIG. 4 shows a side elevation view of the trays of FIG. 1 abutted against one another.

FIG. 5 shows a side elevation view of the bottom tray of FIG. 1.

FIG. 6 shows a side elevation view of the top tray of FIG. 1.

FIG. 7 shows a front and side view of the bottom tray of FIG. 1.

FIG. 8 shows a top view of the top tray of FIG. 1.

### DETAILED DESCRIPTION OF EMBODIMENTS OF THE DISCLOSED TECHNOLOGY

A sleep apnea device having top and bottom trays, as well as a method of use thereof, is disclosed herein. Each tray is adapted to be retained on respective top and bottom teeth, the bottom tray having two flanges extending upwards from a generally planar side of the bottom tray, opposite a side which abuts the teeth. As such, when worn, the flanges extend toward the top tray. The top tray has corresponding flat regions on each side thereof which cut into the outside (relative to the teeth) plane of the tray. The flanges thus abut such flat regions on the outer side of the upper tray, pushing the lower tray and, therefore, the lower teeth and tongue forward. A wearer may then sleep wearing the trays, so as to prevent obstruction of the airways by the tongue during sleep. The pads covering the teeth provide a biting surface that is different that the normal bite. This can be used to treat sleep bruxism concurrently to OSA treatment. The opposing surfaces provide a stable, flat biting surface to treat sleep bruxism. These surfaces may be modified and refined to provide a muscle balanced position which discourages sleep bruxism.

Embodiments of the disclosed technology will become clearer in view of the foregoing description of the figures. Before delving into the figures, it should be understood that “top,” “bottom,” “lower,” “upper,” “front,” and “back” are directional terms relative to the typical order of placement of the trays of the oral appliance relative to one another and their orientation in a mouth. Thus, the “upper tray” fits the upper teeth, “lower tray” fits the lower teeth, “front” refers to the side near the front of the mouth, and so forth.

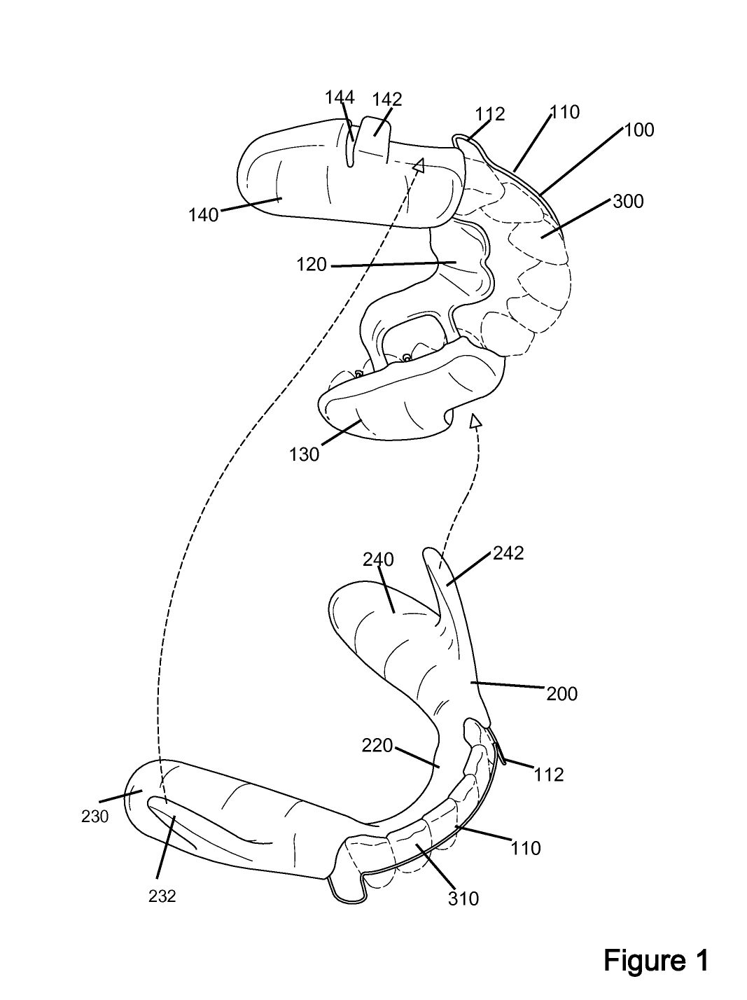

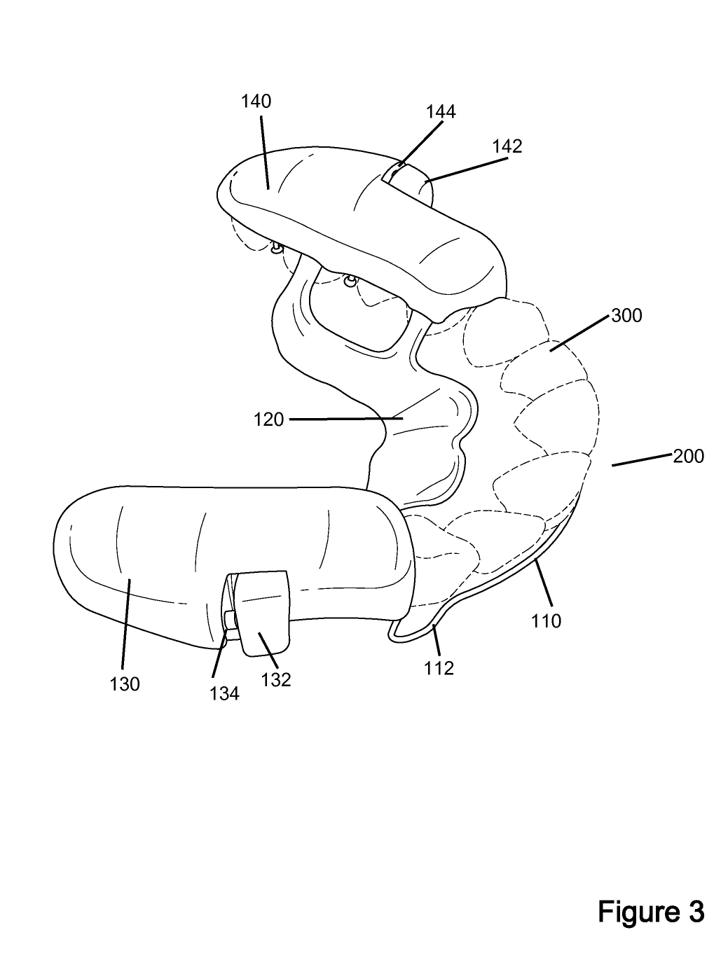

FIG. 1 shows a lower perspective view of an upper tray and upper perspective view of a lower tray to show how the two trays fit together in an embodiment of the disclosed technology. The oral appliance has two distinct parts, a separate upper tray 100 and lower tray 200. The upper tray has two mounting regions 140 and 130 (see also FIG. 3), the region 130 on an opposite side of region 140. Each end region 130 and 140 is adapted to, and defined as, a portion which comprises a mounting region and abuts all molars and pre-molars. In embodiments, the mounting region abuts a plurality of teeth on the outer, inner, and lower portions thereof. A connector, such as a wire 110 (made, in embodiments, from stainless steel) and/or plate 120 (made, in embodiments, from cast chrome cobalt), connects the two mounting regions 130 and 140 and extends through regions 130 and 140 to add additional strength. As shown in FIG. 1, the wire and mounting/end regions (130 and 140), the ball clasps or clips, as well as the wire loop 112, serve to position the upper tray on the upper teeth 300 in a fixed position relative to the teeth. Compared to prior retainers known in the art, this upper tray may be looser to be more comfortable, as the lower tray 200 is pressed against the upper tray, in embodiments of the disclosed technology. This increases compliance through comfort.

Still referring to the upper tray 100, the mounting region further comprises a gap 144 and a generally flat front portion/adjustable block 142. The term “generally” is defined as “according to what an average observer would call as such.” Thus, “generally flat” is defined as what an average person would call “flat” or within a 5% tolerance level of geometrically flat. The front portion/adjustable block 142 comprises a generally perpendicular portion (that is, defined as perpendicular to the plane of the mounting piece from which the front portion extends). Such a generally perpendicular portion may be on a front and back side of the front portion 142, forming the space 144 on one side thereof and an abutment region on the other side thereof. As such, the front portion, in embodiments of the disclosed technology, is adapted for abutment to the lower tray, pushing into the front side of the front portion. The back side of the front portion faces towards a gap, which gap may be opened and closed by way of a screw, clip, wire with key for turning, or other attachment mechanism to adjust the distance from the rear-most portion of the tray to the front-most portion.

Now referring to the lower tray, still viewing FIG. 1, the lower tray has at least two mounting regions 230 and 240, defined in the same manner as the mounting regions 130 and 140 of the upper tray, except adapted to abut lower teeth 310. with adjoined outer, inner, and upper portions adapted to abut corresponding portions of lower teeth. A central region 220 situated between mounting regions connects the two mounting regions and may have a wire 210 and/or continuous chrome cobalt, other metal, or otherwise formed tray piece 220. Further, the bottom tray 200 has a flange 232 and 242 extending from each of the two respective mounting regions 230 and 240 of the lower tray 200, generally away from the lower teeth. That is, the flanges 232 and 242 extend towards the upper tray. Each flange 232 and 242 is identical in an embodiment of the disclosed technology and, in embodiments, has a cast chrome cobalt mesh extending into it. Further, each tray may have a line of symmetry between the right and left sides thereof.

Still referring to the flanges 232 and 242, each front and back side may be angled, but not necessarily to the same degree. As shown in the figures, the front side of the flanges 232 and 242 are angled more than the back side. The back side of each flange is adapted to abut a corresponding front side of a front portion/adjustable block 132 or 142 of the upper tray 100. In embodiments, an angle of extension of one or both flanges 132 and/or 142 is between 60 and 80 degrees, relative to the length of the mounting region which extends over the adjacent sides of the plurality of lower teeth. This angle may be any one of 90 degrees, 85 degrees, 80 degrees, 75 degrees, 70 degrees, 65 degrees, 60 degrees, or 55 degrees. For purposes of this disclosure, any such extension angle between and including 45 degrees and 85 degrees is considered to be “diagonal,” and the extension angle may be defined by either the front side angle or the back side angle of one or both flanges 132 and/or 142.

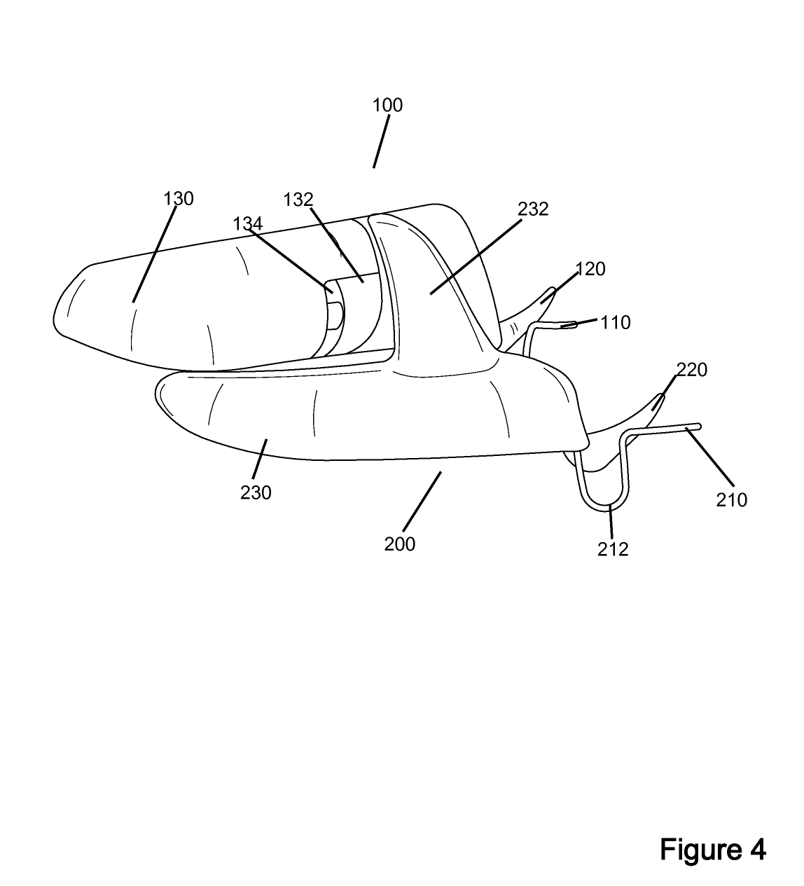

Skipping to FIG. 4, this figure shows a side elevation view of the trays of FIG. 1 abutted against one another. The front side (side closest to the opening of the mouth) is on the right, with back side on the left, in this figure. Here, a flange 232, extending upwards from the (generally) planar surface at the top of the rest of the lower tray 200 has a back side of the flange abutting a front side of the front portion 132 of the upper tray 100. On the reverse side, the flange 242 abuts the front side of the front portion 142 of the upper tray 100.

Note that, as shown in FIG. 4, the retaining trays are aligned such that the bottom tray 200 is forward of the top tray 100. The flanges 232 and 242 are abutted against outer sides (facing away from the teeth) of the mounting regions 130 and 140, causing the lower teeth to be pushed forward relative to the upper teeth which are pushed back. This causes the lower jaw to be held forward, from its resting position and from its position, which would allow the upper and lower teeth of a person to close, such that the upper and lower teeth, or most of them, touch one another. Still further, by pushing the lower jaw forward relative to the upper jaw, the tongue is held forward. In obstructive sleep apnea, the tongue falls backward, obstructing air passage from the mouth to the throat. By holding the jaw forward, and thus, the tongue forward, this prevents the tongue from falling backward. The tongue is pushed forward over the bottom teeth and front side of the bottom tray 200, towards or onto the lower lip. The device is open in the front region allowing the tongue to sit forward without restrictions. This further advances the tongue out of the throat region.

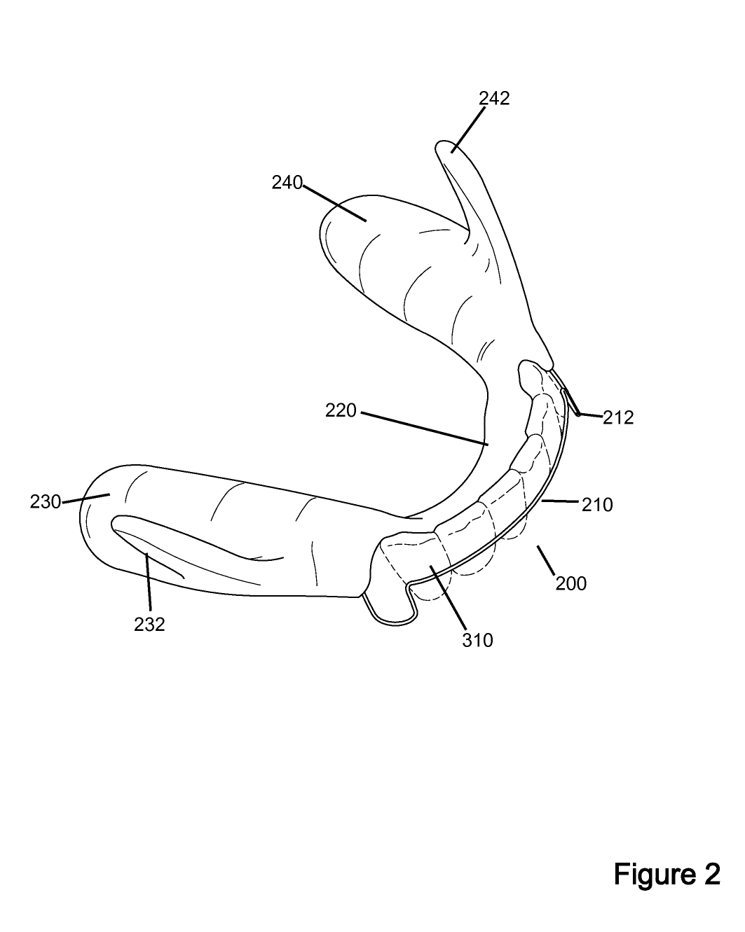

FIG. 2 shows a top perspective view of the bottom tray of FIG. 1. This is the side of the bottom tray 200 which abuts the top tray 100. The flanges 232 and 242 are shown on either side, as well as the position of the upper teeth 200. The bottom of the mouth corresponds to the bottom of this figure.

FIG. 3 shows a bottom perspective view of the top tray of FIG. 1. This is the side of the top tray 100 which abuts the bottom tray 200. The top of the figure corresponds with the bottom of the mouth, when inserted, the upper teeth 300 shown from the bottom. The mounting regions 130 and 140 are shown along with front portions of 132 and 142.

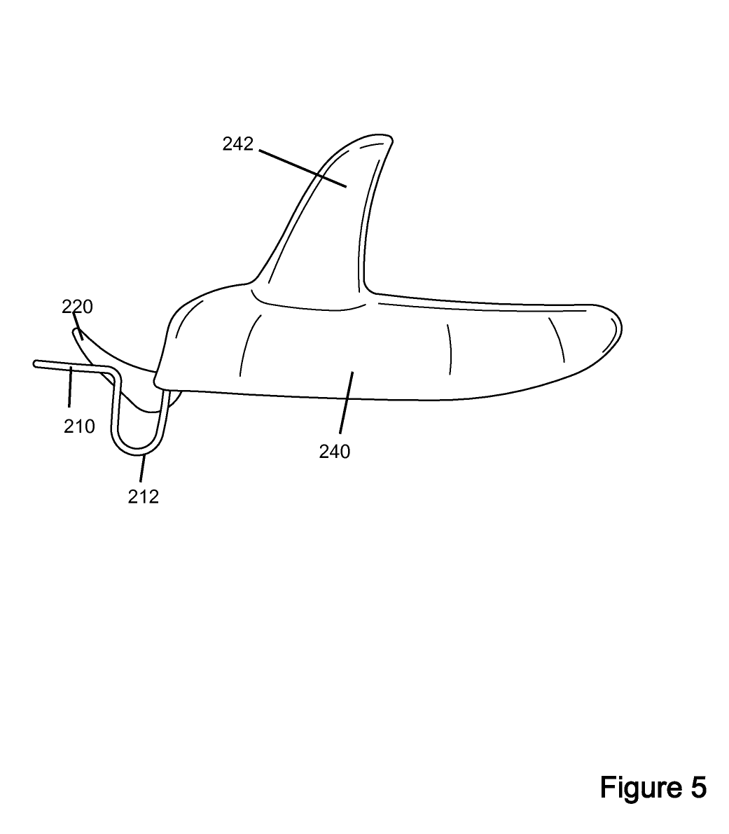

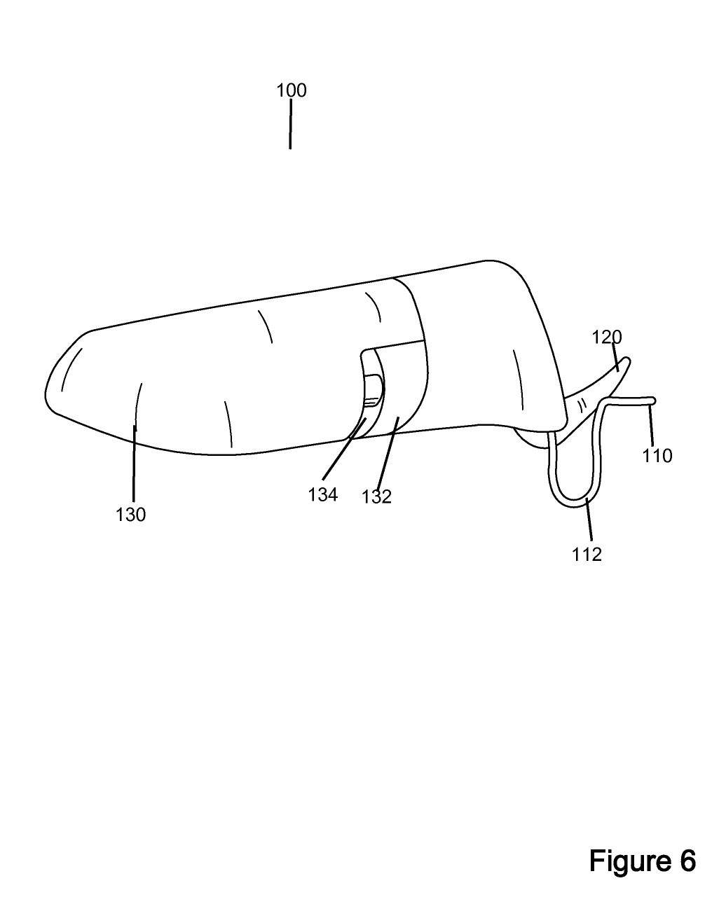

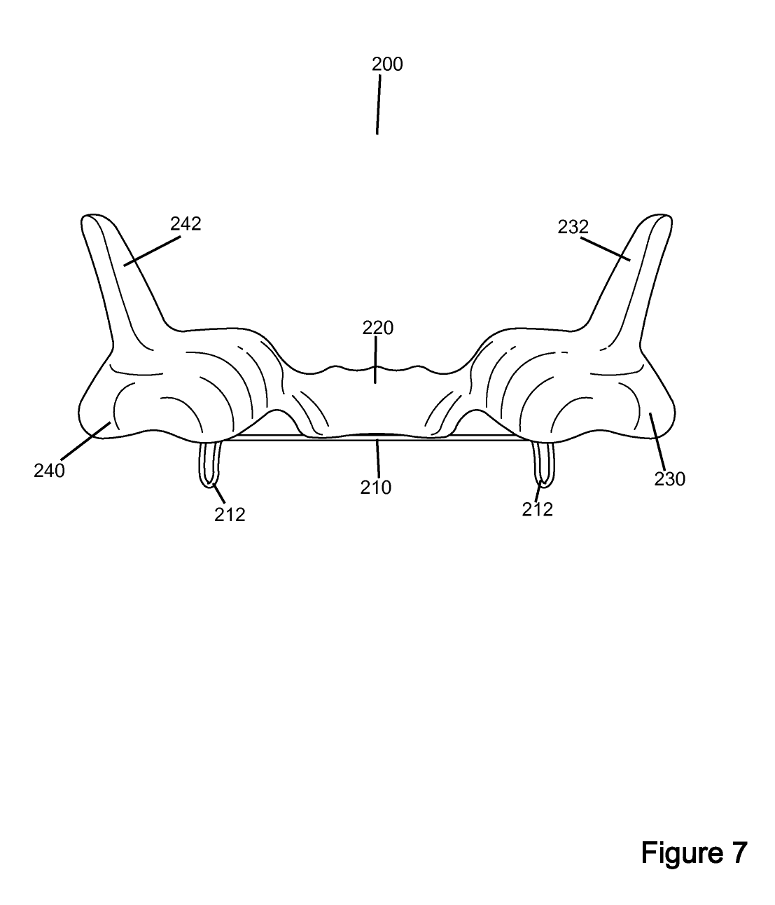

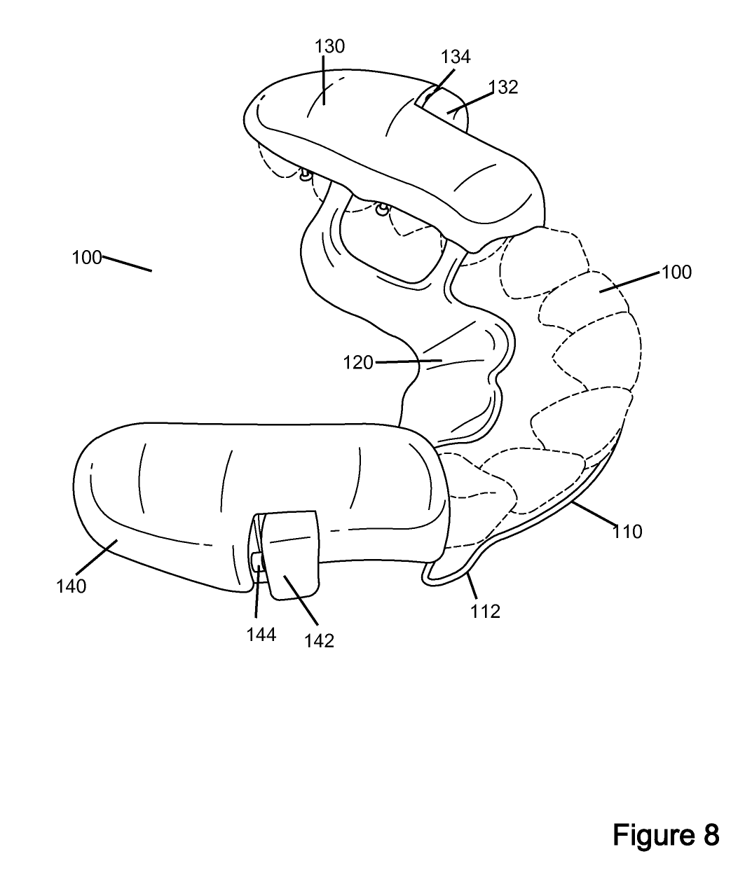

FIG. 5 shows a side elevation view of the bottom tray of FIG. 1. FIG. 6 shows an inverted side elevation view of the top tray of FIG. 1. FIG. 7 shows a front rear view of the bottom tray of FIG. 1. FIG. 8 shows a top view of the top tray of FIG. 1.

In a method of treating obstructive sleep apnea with a two-piece oral appliance, using the device described herein-above, one dons the lower tray 200 with an inner surface thereof abutting lower teeth 310, and an outer surface opposite the inner surface having two flanges 232 and 242 extending upwards from the outer surface. One also (before or after) dons the upper tray 100, this tray having an inner surface abutting upper teeth 300, and an outer surface opposite the inner surface, the outer surface being generally planar and interrupted by a surface which is generally perpendicular to said generally planar surface, forming the area designated as 132 and 142, in the figures. Then, one abuts the two flanges 232 and 242, each to a generally perpendicular surface at the front of area 142, on the outer surface of the upper tray 100. This abutting of upper and lower trays causes the lower teeth to be held in front of the upper teeth, relative to a resting position of the teeth. One then sleeps with the appliance on the teeth.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods, systems, and devices described hereinabove are also contemplated and within the scope of the invention.

I claim:

1. An oral appliance, comprising:
separate upper and lower trays,
said upper tray comprising at least two mounting regions with adjoined outer, inner, and lower portions adapted to abut corresponding portions of upper teeth;
each said outer portion of each said mounting region of said upper tray further comprising a generally flat front portion, a generally perpendicular portion, to said flat front portion forming a corner there-between, and a back portion extending from said side perpendicular to said flat front portion;
said lower tray comprising at least two mounting regions with adjoined outer, inner, and upper portions adapted to abut corresponding portions of lower teeth;
a flange extending from each of said two mounting regions of said lower tray, generally away from said lower teeth.

2. The oral appliance of claim 1, wherein said flange is adapted to abut said generally perpendicular portion of said mounting region of said upper tray.

3. The oral appliance of claim 2, wherein, compared to a resting position of said teeth, said bottom teeth are forced forward, relative to said bottom teeth, when said flange abuts said generally perpendicular portion of said mounting region of said upper tray.

4. The oral appliance of claim 3, wherein said tongue is held forward, compared to its resting position.

5. The oral appliance of claim 1, wherein said lower side of said mounting region of said upper tray, and said upper side of said mounting region of said lower tray are adapted to abut one another along a length thereof, extending over respective adjacent sides of a plurality of teeth when said flange abuts said generally perpendicular portion of said mounting region of said upper tray.

6. The oral appliance of claim 5, wherein said flange extends diagonally upwards from said lower tray, to the outside of said upper tray.

7. The oral appliance of claim 6, wherein the angle of extension of said flange is between 60 and 80 degrees, relative to said length of said mounting region which extends over said adjacent sides of said plurality of teeth.

8. The oral appliance of claim 1, wherein said side perpendicular to said flat portion of said upper tray further forms a side of an adjustable block further comprising an adjustable length attachment, adjustably attaching said adjustable block to a rear portion of said mounting portion of said upper tray such that said adjustable block is movable closer to said rear portion by way of said adjustable length attachment.

9. The oral appliance of claim 8, wherein said adjustable length attachment is a screw rotatable by inserting a wire key.

10. The oral appliance of claim 8, wherein said flange abuts both said adjustable block and said generally flat front portion of said upper tray, when worn by a user.

11. The oral appliance of claim 1 wherein, for each tray, said mounting regions are connected to one another via a wire, said wire adapted to abut front sides of teeth situated between said mounting regions.

12. A method of treating obstructive sleep apnea with a two piece oral appliance, comprising the steps of:
donning a lower tray of said oral appliance with a cast chrome cobalt inner surface abutting lower teeth, and an outer surface opposite said inner surface, said outer surface further comprising two flanges extending upwards from said outer surface;
donning a upper tray of said oral appliance with an inner surface abutting upper teeth, and an outer surface opposite said inner surface, said outer surface being generally planar and interrupted by a surface which is generally perpendicular to said generally planar surface;
abutting said two flanges, each to a said generally perpendicular surface of said outer surface of said upper tray.

13. The method of claim 12, wherein said abutting causes said lower teeth to be held in front of said upper teeth, relative to a resting position of said teeth.

14. The method of claim 13, wherein a wearer of said two piece oral appliance sleeps with said oral appliance donned on said teeth.

15. A two piece oral appliance device for treating obstructive sleep apnea, comprising:
a lower tray of said oral appliance with an inner surface abutting lower teeth, and an outer surface opposite said inner surface, said outer surface further comprising two flanges extending upwards from said outer surface;
an upper tray of said oral appliance with an inner surface abutting upper teeth, and an outer surface opposite said inner surface, said outer surface further generally planar and interrupted by a surface which is generally perpendicular to said generally planar surface;
wherein each of said two flanges is adapted to abut a said generally perpendicular surface of said outer surface of said upper tray.

16. The device of claim 15, wherein said abutting causes said lower teeth to be held in front of said upper teeth, relative to a resting position of said teeth.

17. The device of claim 15, wherein said flange is adapted to abut said generally perpendicular portion of said outer surface of said upper tray.

18. The device of claim 15, wherein said flange extends diagonally upwards from said lower tray, to the outside of said upper tray.

19. The device of claim 17, wherein said perpendicular to said flat portion of said upper tray further forms a side of an adjustable block further comprising an adjustable length attachment, adjustably attaching said adjustable block to a rear portion of said mounting portion of said upper tray, such that said adjustable block is movable closer to said rear portion by way of said adjustable length attachment.

20. The device of claim 19, wherein said lower and said upper trays are worn, a lower jaw and tongue are pushed forward, compared to a resting position thereof.
